(19) United States
(12) Patent Application Publication
(10) Pub. No.: US 2015/0250713 A1
(43) Pub. Date: Sep. 10, 2015

(54) Pharmaceutical Composition

(71) Applicant: CIPLA LIMITED, Mumbai (IN)

(72) Inventors: Geena Malhotra, Mumbai (IN); Shrinivas Purandare, Mumbai (IN)

(73) Assignee: CIPLA Limited, Mumbai (IN)

(21) Appl. No.: 14/431,561

(22) PCT Filed: Oct. 23, 2013
(86) PCT No.: PCT/GB2013/000454

§ 371 Date: Mar. 26, 2015

(30) Foreign Application Priority Data
Oct. 23, 2012 (IN) 3092/MUM/2012
Jan. 31, 2013 (IN) 280/MUM/2013

(51) Int. Cl. A61K 9/00; A61K 47/06; A61K 31/46

(52) U.S. Cl. CPC A61K 9/008; A61K 31/46; A61K 47/06

(57) ABSTRACT

The present invention relates to a pharmaceutical composition comprising tiotropium, a hydrofluoroalkane (HFA) propellant, and optionally one or more pharmaceutically acceptable excipients; to a process for preparing such a pharmaceutical composition, and the use thereof in medicine, in particular for the prophylaxis and treatment of respiratory disorders.

### FIELD OF THE INVENTION

The present invention relates to a stable pharmaceutical composition comprising tiotropium with at least one hydrofluoroalkane (HFA) propellant. The present invention also relates to the process of preparing the same and its use for the treatment of asthma, COPD and other respiratory disorders thereof.

### BACKGROUND AND PRIOR ART

Chronic obstructive pulmonary disease (COPD) is a severe respiratory condition that is increasing its prevalence worldwide. In India, the estimated prevalence is about 12.36 million. It is currently the fourth leading cause of death in the UK & US and is predicted to rank third in the “global impact of disease” by the year 2020.

Chronic obstructive pulmonary disease (COPD) is a preventable and treatable disease state characterized by air flow limitation that is not fully reversible. The airflow obstruction is usually progressive and associated with an abnormal inflammatory response of the lungs to noxious particles or gases primarily caused by cigarette smoking. Although COPD affects the lungs, it also produces significant systemic consequences. COPD is associated with mucus hyper secretion, emphysema and bronchiolitis.

Asthma is a major cause of chronic morbidity and mortality with an estimated 300 million affected individuals worldwide and 250,000 annual deaths are attributed to the disease. People of all ages in most countries are affected by this chronic disease.

Asthma is a chronic inflammatory disorder of the airways associated with airway hyper responsiveness that leads to recurrent episodes of wheezing, breathlessness, chest tightness and coughing. An increased inflammatory response is a major part of the pathophysiology of acute asthma and regular preventive treatment is important.

The major goals of the therapy for the prevention and treatment of COPD, asthma and other respiratory disorders include smoking cessation, relief of symptoms, improvement in physiological functions and limiting complications such as abnormal gas exchange and exacerbation of disease. However, an integrated approach to the treatment involves the combination of healthcare maintenance such as smoking cessation, avoidance of indoor as well as outdoor pollutants and allergens, avoidance of occupational exposure to allergens and use of drugs and supplemental therapies in a step-wise fashion as the disease progresses.

Currently, the therapy for the treatment or prevention of COPD and asthma includes the use of one or more long acting bronchodilators such as β2 agonists, anticholinergics, inhaled corticosteroids (ICS) or combinations thereof.

Tiotropium bromide is one such anticholinergic bronchodilator that antagonises the M1, M2 and M3 muscarinic receptors. Tiotropium is chemically known as (1α, 2β, 4β, 5α, 7β)-7-[(Hydroxydi-2-thienylacetyl)oxy]-9, 9-dimethyl-3-oxa-9-azoniatricyclo [3.3.1.02,4] nonane bromide monohydrate and is represented as:

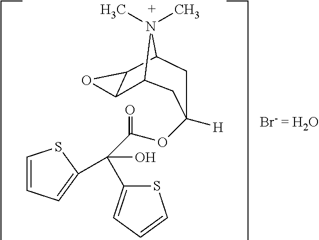

Tiotropium has a longer duration of action of up to 32 hours. Also, tiotropium exhibits an improvement in dyspnea and ceases the need for rescue therapy. Tiotropium in combination with pulmonary rehabilitation (PR) associated with an increased exercise endurance time produces clinically meaningful improvements in dyspnea and health status as compared to pulmonary rehabilitation alone in COPD patients.

Tiotropium is an extremely moisture sensitive drug. Therefore, formulations containing tiotropium undergo hydrolytic degradation which results in an unstable formulation thus deteriorating its required efficacy.

Several attempts have been made by formulators to stabilize such formulations of tiotropium by adding additional excipients such as mineral acids, lower branched or linear alkyl (C1-C4) alcohols and active adjuvant complexes.

For example, EP2201934 discloses stable tiotropium HFA aerosol solution formulations wherein stability is achieved by addition of a mineral acid such as hydrochloric, phosphoric, nitric and sulphuric acid in the formulation. However, to improve the stability, it is not only necessary to add small amounts of mineral acids but it is also important to select the type of the container, metering valve and sealing gaskets which may be in contact with the formulation. Further, the addition of mineral acids may increase the toxicity of the formulation.

WO2010052466 discloses a stable pharmaceutical aerosol composition of tiotropium complexed with an adjuvant (PVP) for the treatment of respiratory disorders.

EP 1870090 discloses a novel stable formulation of suspended aerosols and hydrofluorocarbons as propellants wherein stability is achieved'by adding a dispersion coadjuvant such as ethanol in very small quantities (lower than 0.30% w/w) and the use of valves provided with seals compatible with the formulation. However, by increasing or decreasing the quantity of ethanol, the values of FPF (Fine Particle Fraction) and MMAD (Mass Median Aerodynamic Diameter) would vary drastically.

Although the above prior arts cover various techniques to prepare stable formulations of tiotropium, the use of excipients in lower quantities, specific types of containers and valves may limit the ease of the manufacture and vary the cost.

Considering the above limitations, there still arises a need to develop a pharmaceutical composition of tiotropium which can be produced by simple manufacturing techniques and which also exhibit improved stability.

Although the use of HFA propellants has been very well disclosed in the prior art, the inventors have surprisingly found that compositions comprising tiotropium and specifically HFA-227 as a propellant, exhibit improved stability as compared to other HFA propellants, particularly HFA-134 (a).

### OBJECT OF THE INVENTION

An object of the present invention is to provide a pharmaceutical composition comprising tiotropium and a HFA propellant, such as HFA-227, optionally with one or more pharmaceutically acceptable excipients.

Another object of the present invention is to provide a pharmaceutical composition comprising tiotropium and a HFA propellant, such as HFA-227, optionally with one or more pharmaceutically acceptable excipients, which composition exhibits improved stability.

Yet another object is to provide a process for the preparation of a pharmaceutical composition comprising tiotropium and a HFA propellant, such as HFA-227, optionally with one or more pharmaceutically acceptable excipients.

Another object of the present invention is to provide a method for prophylaxis or treatment of asthma, chronic obstructive pulmonary disease or related respiratory disorders by administering a pharmaceutical composition comprising tiotropium and a HFA propellant, such as HFA-227, optionally with one or more pharmaceutically acceptable excipients.

Another object of the present invention is to provide the use of a pharmaceutical composition comprising tiotropium and a HFA propellant, such as HFA-227, optionally with one or more pharmaceutically acceptable excipients for the treatment of asthma and chronic obstructive pulmonary disease or related respiratory disorders.

### SUMMARY OF THE INVENTION

According to a first aspect of the present invention, there is provided a pharmaceutical composition comprising tiotropium, preferably tiotropium bromide and a WA propellant, preferably HFA-227, optionally with one or more pharmaceutically acceptable excipients.

According to another aspect of the present invention, there is provided a pharmaceutical composition comprising tiotropium optionally with one or more pharmaceutically acceptable excipients comprising HFA-227 as a propellant. Preferably, the composition is formulated for administration using a metered dose inhaler (MDI).

According to another aspect of the present invention, there is provided a pharmaceutical composition comprising tiotropium and another active ingredient, preferably a beta adrenergic agonist and/or an inhaled corticosteroid, a HFA propellant, preferably HFA-227, optionally with one or more pharmaceutically acceptable excipients. Preferably, the composition is formulated for administration using a metered dose inhaler (MDI).

According to another aspect of the present invention, there is provided a process for the preparation of a pharmaceutical composition, such process comprising admixing tiotropium along with a HFA propellant, preferably HFA-227, optionally with one or more pharmaceutically acceptable excipients.

According to another aspect of the present invention, there is provided a method for prophylaxis or treatment of asthma, chronic obstructive pulmonary disease or related respiratory disorders by administering a pharmaceutical composition comprising tiotropium and a HFA propellant, such as HFA-227 optionally along with pharmaceutically acceptable excipients.

According to another aspect of the present invention, there is provided the use of a pharmaceutical composition comprising tiotropium and a HFA propellant, such as HFA-227, optionally along with pharmaceutically acceptable excipients for the prophylaxis or treatment of asthma, chronic obstructive pulmonary disease or related respiratory disorders.

According to another aspect of the present invention, there is provided the use of a pharmaceutical composition comprising tiotropium and a HFA propellant, such as HFA-227, optionally along with pharmaceutically acceptable excipients in the manufacture of a medicament for the prophylaxis or treatment of asthma, chronic obstructive pulmonary disease or related respiratory disorders.

### BRIEF DESCRIPTION OF THE DRAWINGS

Studies were carried out on pharmaceutical compositions comprising tiotropium, a HFA propellant (either HFA-227 or HFA 134 (a)) and lactose.

(A) Stability Data

FIG. 1: Comparative stability data of tiotropium with propellant HFA 134(a) versus tiotropium with propellant HFA-227.

This figure indicates that the drop from the initial % L.A. (Labelled Amount)-value to the 6 month % L.A. value is substantially lower in the case of tiotropium with propellant HFA-227 as compared to tiotropium with propellant HFA 134(a). This indicates that the tiotropium HFA-227 composition is more stable than the tiotropium HFA 134(a) composition.

(B) Particle size distribution studies replicating the deposition of particles in the human lung.

FIG. 2: Particle size distribution study of propellant HFA 134(a) using a Cascade Impactor.

FIG. 3: Particle size distribution study of propellant HFA-227 using a Cascade Impactor.

Stages S3, S4 and S5 are the critical stages which indicate the deep lung deposition of tiotropium.

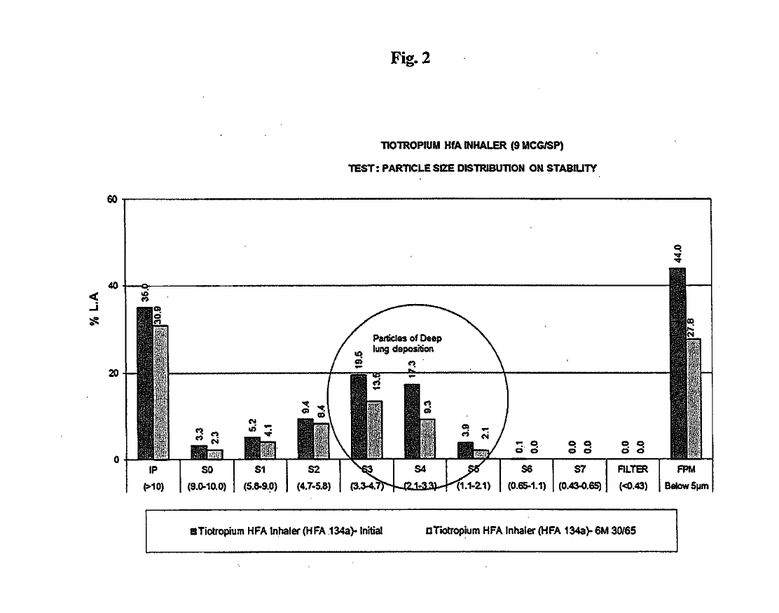

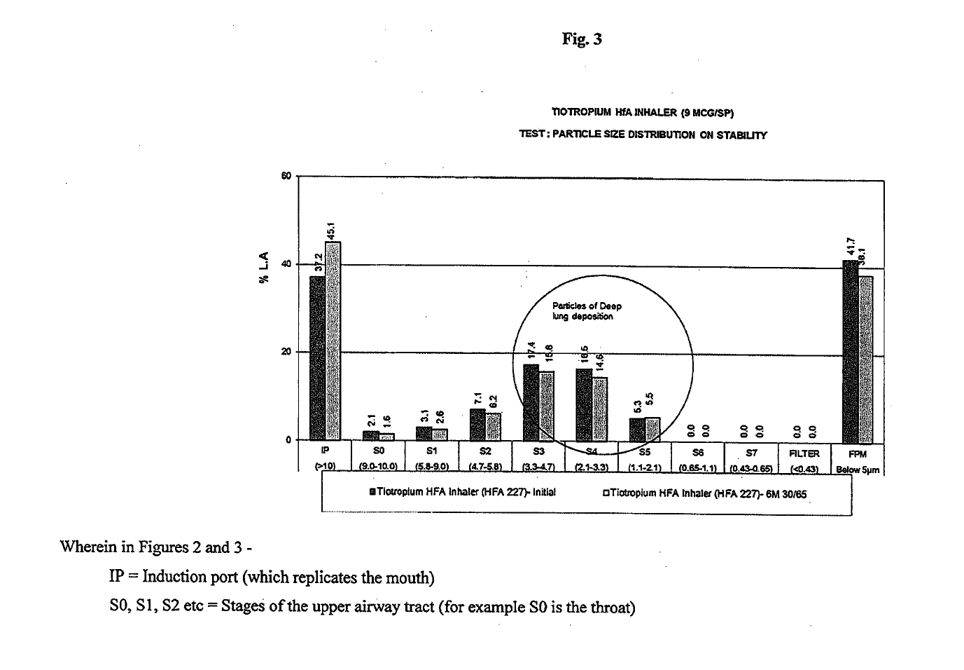

FIGS. 2 and 3 illustrate that the drop from the initial % L.A value to the 6 month % L.A value for the tiotropium HFA-227 composition at both the S3 and S4 stage is less than for the tiotropium 134(a) composition. This indicates that the tiotropium HFA-227 composition is more stable than the tiotropium HFA 134(a) composition.

### DETAILED DESCRIPTION OF THE INVENTION

The inventors of the present invention have developed a pharmaceutical composition comprising tiotropium and a HFA propellant, preferably HFA-227, optionally with one or more pharmaceutically acceptable excipients.

The present invention provides a process for the preparation of such pharmaceutical compositions and also provides their use in the treatment of asthma, chronic obstructive pulmonary disease or any other related respiratory disorders.

The pharmaceutical compositions of the present invention are preferably aerosol compositions for administration using a metered dose inhaler (MDI) or the like.

Further, such pharmaceutical compositions may comprise tiotropium and additional actives, a propellant and optionally one or more excipients. The active ingredient can be selected from the group comprising beta adrenergic agonists and/or inhaled corticosteroids.

As used herein the terms “tiotropium, albuterol, salbutamol, levoalbuterol, levosalbutamol butaline, pirbuterol, procaterol, metaproterenol, fenoterol, isoproterenol (β₁ and β₂), metaproterenol, terbutaline, isoetarine, bitolterol mesylate, ritodrine, salmeterol, formoterol, arformoterol, carmoterol, bambuterol, clenbuterol, indacaterol, milveterol, vilanterol, olodaterol, fluticasone propionate, fluticasone furoate, fluticasone valerate, mometasone, ciclesonide, beclomethasone, budesonide, R-budesonide” are used in a broad sense to include not only the active ingredient per se but also pharmaceutically acceptable derivatives thereof. Suitable derivatives include pharmaceutically acceptable salts, pharmaceutically acceptable solvates, pharmaceutically acceptable hydrates, pharmaceutically acceptable isomers, pharmaceutically acceptable esters, pharmaceutically acceptable anhydrates, pharmaceutically acceptable enantiomers, pharmaceutically acceptable polymorphs, pharmaceutically acceptable prodrugs, pharmaceutically acceptable tautomers and/or pharmaceutically acceptable complexes thereof, or any combination of such derivatives. A preferred pharmaceutically acceptable salt of tiotropium is tiotropium bromide, preferably tiotropium bromide monohydrate or tiotropium bromide anhydrate.

Chlorofluorocarbons (CFC) were previously the most common propellants used in pressurised Metered Dose Inhalers (pMDIs) due to the fact that they are non-toxic, non-flammable and have high vapor pressure. However CFC's, as propellants, have adverse effects on global warming and stratospheric ozone destruction due to which the use of the same has been banned in many countries, which led to the extensive usage of HFA as propellant systems.

Thus, two new hydrofluoroalkanes (HFAs) namely tetrafluoroethane (HFA-134a) and heptafluoropropane (HFA-227), have become the alternative propellants for use with pharmaceutical aerosols delivered in pMDIs. HFAs also have similar advantages to CFCs for use in pMDIs and they also do not cause any damage to the ozone layer.

The HFA propellant must be toxicologically safe and must have a vapor pressure which is suitable to enable the medicament to be administered via a pressurized MDI. Additionally, the HFA propellant must be compatible with the components of the MDI device (such as containers, valves, and sealing gaskets, etc.) which are employed to administer the medicament. Examples of suitable HFA propellants for use in the present invention are HFA-32 (difluoromethane), HFA-143(a) (1,1,1 -trifluoroethane), HFA-134 (1,1,2,2-tetrafluoroethane), and HFA-152a (1,1-difluoroethane), 1,1,1,2-tetrafluoroethane (HFA-134(a)) and 1,1,1,2,3,3,3,-heptafluoropropane (HFA-227), and any mixture thereof. Preferred HFA propellants are 1,1,1,2-tetrafluoroethane (HFA-134(a)) and 1,1,1,2,3,3,3,-heptafluoropropane (HFA-227) or a mixture thereof. The terms hydrofluoroalkane (HFA) and hydrofluorocarbon (HFC) are used interchangeably throughout this specification.

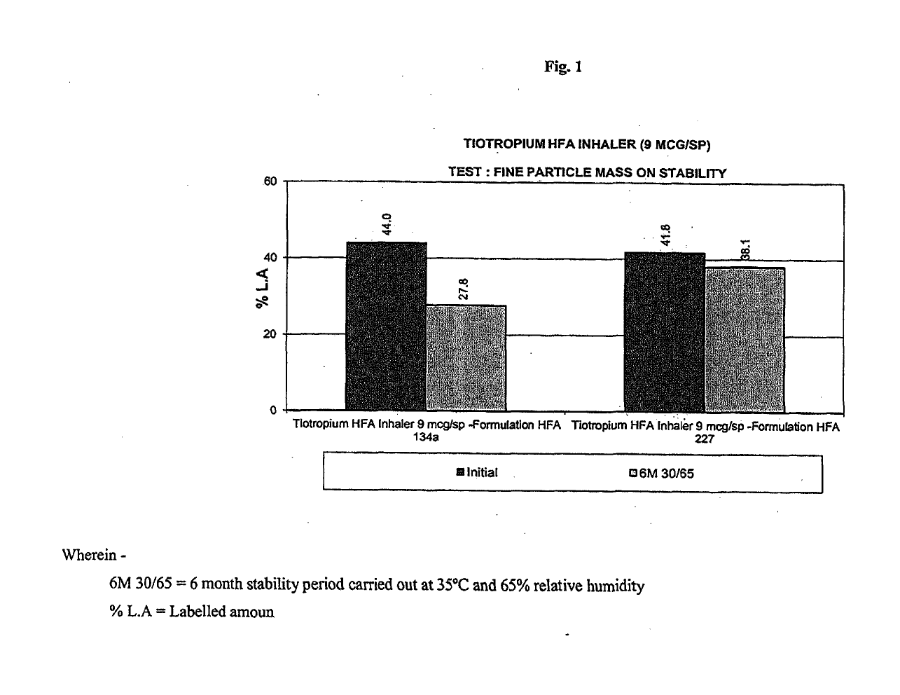

The inventors of the present invention have found that the use of HFA-227 specifically as a propellant system resulted in a more stable pharmaceutical composition of tiotropium when compared with a pharmaceutical composition of tiotropium comprising HFA-134 (a) as a propellant system. This is illustrated in FIGS. 1, 2 and 3.

The moisture uptake of HFA-134 (a) is six times higher compared to HFA-227 (measured values) due to its higher polarity. This moisture uptake may increase the particle size of tiotropium leading to a lower fine particle dose (FPD) value which in turn causes lesser bioavailability.

Further, HFA-134 (a) causes the cold Freon effect which may ultimately affect patient compliance.

Suitable excipients may be optionally used for formulating the pharmaceutical composition according to the present invention. Examples of suitable pharmaceutically acceptable excipients may comprise one or more, but not limited to, HFA propellants, non-halogenated hydrocarbon propellants, co-solvents, low volatility components, stabilizers, dispersing agents, pH adjusting agents, antioxidants, preservatives, chelating agents, surface active agents, bulking agents and the like or mixtures thereof.

HFA propellants are carriers which, when mixed with the cosolvent(s), form a homogeneous propellant system in which a therapeutically effective amount of the medicament can be dissolved. The HFA propellant must be toxicologically safe and must have a vapor pressure which is suitable to enable the medicament to be administered via a pressurized MDI. Additionally, the HFA propellant must be compatible with the components of the MDI device (such as containers, valves, and sealing gaskets, etc.) which are employed to administer the medicament. The preferred HFA propellant is 1,1,1,2,3,3,3,-heptafluoropropane (HFA-227). Other examples of suitable HFA propellants are HFA-32 (difluoromethane), HFA-143(a) (1,1,1 -trifluoroethane) and HFA-152a (1,1-difluoroethane) and mixtures thereof.

Non-halogenated hydrocarbon may be used in combination with the HFA propellants of the present invention. Examples of such non-halogenated hydrocarbons are saturated hydrocarbons, including propane, n-butane, and isobutane, and ethers, including diethyl ether and the like or mixtures thereof.

It will also be apparent to those skilled in the art that, although the use of a single HFA propellant is preferred, a mixture of two or more HFA propellants, or a mixture of at least one HFA propellant and one or more non-CFC propellants, may be employed in the composition (aerosol solution formulation) of the present invention.

Suitable co solvents and low volatility components that may be employed to increase the compatibility between the drug and the propellant in the pharmaceutical composition may comprise one or more C₂-C₆ aliphatic alcohols such as, but not limited to, ethyl alcohol and isopropyl alcohol; glycols such as, but not limited to, propylene glycol, polyethylene glycols, polypropylene glycols, glycol ethers, and block copolymers of oxyethylene and oxypropylene; and other substances such as, but not limited to glycerol, isopropyl myristate polyoxyethylene alcohols, and polyoxyethylene fatty acid esters; hydrocarbons such as, but not limited to, n-propane, n-butane, isobutane, n-pentane, iso-pentane, neo pentane and n-hexane; ethers such as but not limited to diethyl ether and the like or mixtures thereof. The co-solvent is preferably present in an amount ranging from 0.1-5% of the composition.

Suitable bulking agents that may be employed in the pharmaceutical composition of the present invention may comprise saccharides such as, but not limited to, monosaccharides, disaccharides, oligosaccharides, and polysaccharides for example lactose, maltose, glucose, fructose, galactose arabinose, dextrose, ribose, sucrose, sorbitol, mannitol, xylose, trehalose, raffinose, melezitose, glycerol, erythritol, xylitol, maltitol, lactitol and D & L series of rare sugars and the like and mixtures thereof The bulking agent is preferably present in an amount ranging from 10-500% of the drug. More preferably, the bulking agent is present in an amount ranging from 10-300%, 50-300%, 50-200% of the drug (for example, tiotropium). Most preferably the bulking agent is present in an amount of 300% of the drug.

Suitably, the preservatives that may be employed in the pharmaceutical composition may be present in a range of 0.00001-0.2%, more preferably 0.01-0.2% of the formulation. The preservative that may be employed in the pharmaceutical composition may comprise one or more of benzalkonium chloride, EDTA, benzoic acid, benzoates such as sodium benzoate and such other preservatives which may be known to the person skilled in the art and the like or mixtures thereof.

Suitably the chelating/complexing agents that may be employed in the pharmaceutical composition may be present in a range of 0.00001-0.2%, more preferably 0.01-0.2% of the formulation. The chelating agents that may be employed in the pharmaceutical composition may comprise edetic acid (EDTA) or one of its known salts thereof, e.g. sodium EDTA or disodium EDTA dihydrate (sodium edetate) and the like or mixtures thereof.

Suitably, the pH adjusting agent that may be employed in the pharmaceutical composition may comprise one or more of organic or inorganic acids such as, but not limited to, citric acid, ascorbic acid, hydrochloric acid, sulfuric acid, nitric acid or phosphoric acid and the like or mixtures thereof. Preferably, the one or more organic or inorganic acids are non-mineral acids, such as citric acid and ascorbic acid. Most preferably, the composition of the present invention does not comprise a mineral acid, in other words it is preferable that the composition is essentially free or free of a mineral acid.

One or more surfactants may be employed to stabilize the pharmaceutical composition and to also provide lubrication to the valve system of the metered dose inhaler. The one or more stabilizers is preferably present in a range of 0.00001-0.5% of the composition, more preferably 0.00001-0.2%, even more preferably 0.001-0.3%, and most preferably 0.001-0.1%. Some of the most commonly employed surfactants may comprise one or more ionic and/or non-ionic surfactants such as salts of stearic acids such as magnesium stearate or esters such as ascorbyl palmitate, isopropyl myristate or tocopherol esters such as oleic acid, sorbitan trioleate, lecithin, isopropyl myristate, tyloxapol, or polysorbates such as polysorbate 80, Polysorbate 20, Polysorbate 40, vitamin E-TPGS or macrogol hydroxystearates such as macrogol-15-hydroxystearate or acetylated monoglycerides like Myvacet 9-45 and Myvacet 9-08, Polyoxyethylene ethers, ethyloleate, glyceryl trioleate, glyceryl monolaurate, glyceryl monooleate, glyceryl monosterate, glyceryl monoricinoleate, cetylalcohol, sterylalcohol, cetyl pyridinium chloride, block polymers, natural oils, sorbitan fatty acid esters such as sorbitan trioleate, polyethoxylated sorbitan fatty acid esters (for example polyethoxylated sorbitan trioleate), sorbimacrogol oleate, synthetic amphotensides (tritons) or ethylene oxide ethers of octylphenolformaldehyde condensation products, phosphatides such as lecithin, polyethoxylated fats, polyethoxylated oleotriglycerides and polyethoxylated fatty alcohols. The surfactants may also be selected from the vast class like oils known in the art such as, but not limited to, corn oil, olive oil, cottonseed oil and sunflower seed oil, mineral oils like liquid paraffin, oleic acid and also phospholipids such as lecithin, or sorbitan fatty acid esters like sorbitan trioleate or Tween 20, Tween 60, Tween 80, polyethylene glycols such as PEG-25, PEG-100, PEG-1000 (preferably in an amount of 0.3% of the total weight of the composition), Glyceryl trioleate, PVP (polyvinylpyrrolidone, e.g. PVP K25 preferably in an amount of 0.001% of the total weight of the composition), citric acid, PFDA (per fluoro-n-decanoic acid) and the like or mixtures thereof.

Suitable dispersing agents that may be employed in the pharmaceutical composition may comprise sorbitan trioleate, oleyl alcohol, oleic acid, lecithin and the like or mixtures thereof.

Suitable antioxidants that may be employed in the pharmaceutical composition may comprise ascorbic acid, α-tocopherol, BHT (butylhydroxytoluene) and BHA (butylhydroxyanisole) and the like or mixtures thereof.

Preferably, the composition is essentially free of a mineral acid and/or the composition does not comprise a tiotropium-adjuvant complex, for example a tiotropium-PVP complex. In an alternative it is preferable that the composition is free of a mineral acid and/or the composition does not comprise a tiotropium-adjuvant complex, for example a tiotropium-PVP complex.

Preferably, there is provided a pharmaceutical composition comprising tiotropium bromide, more preferably tiotropium bromide monohydrate or tiotropium bromide anhydrate, and HFA-227. The dose of tiotropium bromide, preferably tiotropium bromide monohydrate, is preferably 9 micrograms (mcg).

The pharmaceutical composition according to the present invention, may further comprise one or more active agents selected from beta adrenergic agonists such as, but not limited to, albuterol or salbutamol, levoalbuterol, levosalbutamol butaline, pirbuterol, procaterol, metaproterenol, fenoterol, isoproterenol (β₁ and β₂), metaproterenol, terbutaline, isoetarine, bitolterol mesylate, ritodrine, salmeterol, formoterol, arformoterol, carmoterol, bambuterol, clenbuterol, indacaterol, milveterol, vilanterol, olodaterol, or inhaled corticosteroids such as, but not limited to, fluticasone propionate, fluticasone furoate, fluticasone valerate, mometasone, ciclesonide, beclomethasone, budesonide, R-budesonide.

According to a preferred embodiment of the present invention, the pharmaceutical composition comprises tiotropium, preferably tiotropium bromide, more preferably tiotropium bromide monohydrate or tiotropium bromide anhydrate, and is formulated for delivery using a pressurized metered dose inhaler. Preferably the tiotropium concentration corresponds to single doses ranging from about 2.5 micrograms to about 18 micrograms, preferably from about 2.5 to about 15 micrograms, more preferably from about 4.5 to about 9 micrograms, characterized by a desirable FPD of the said active particles/ aerosol particles. Most preferably, the composition comprises tiotropium bromide, preferably tiotropium bromide monohydrate at a concentration of about 9 micrograms.

According to another preferred embodiment of the present invention, the pharmaceutical composition comprises tiotropium, such as tiotropium bromide, preferably tiotropium bromide monohydrate or tiotropium bromide ayhydrate, and is formulated for delivery/administration using a metered dose inhaler or a breath actuated metered dose inhaler.

According to another embodiment of the present invention, there is provided a method of administering tiotropium, preferably tiotropium bromide, more preferably tiotropium bromide monohydrate or tiotropium bromide anhydrate, with one or more pharmaceutically acceptable excipients comprising a HFA propellant, preferably HFA-227, co-solvent, low volatility component, stabilizer, dispersing agent, pH adjusting agent, surface active agent or mixtures thereof, to be used with a metered dose inhaler or a breath actuated metered dose inhaler comprising a metering valve and low orifice actuator ranging from 0.2 mm to 0.6 mm diameter (preferably 0.4 to 0.5 mm) characterized by a desirable FPD of the said active particles/aerosol particles.

The pharmaceutical composition according to the present invention may be dispensed in plain aluminum cans or SS (stainless steel) cans. The inner surface of these cans can be coated with suitable polymers. Such polymers include, but are not limited to, fluorocarbon copolymers such as FEP-PES (fluorinated ethylene propylene and polyethersulphone), PFA-PES (perfluoroalkoxyalkane and polyethersulphone), epoxy and ethylene or combinations thereof. Alternatively, the inner surfaces of the cans may be anodized also.

The present invention also provides a process for preparing the pharmaceutical composition of the present invention which process comprises admixing tiotropium with a HFA propellant. Preferably, the admixing step comprises admixing one or more pharmaceutically acceptable excipients with tiotropium, and/or with a HFA propellant.

The present invention also provides a method for the treatment in a mammal, such as a human, for treating chronic obstructive pulmonary disease and asthma, which method comprises administration of a therapeutically effective amount of pharmaceutical compositions according to the present invention.

The method of treatment may be characterized in that the pharmaceutical compositions according to the present invention are administered once or twice a day in therapeutically effective amounts.

In one aspect, the present invention provides a pharmaceutical composition which may further comprise one or more active agents selected from beta adrenergic agonists and/or inhaled corticosteroid for treating chronic obstructive pulmonary disease and asthma.

Accordingly, the present invention provides a pharmaceutical composition which may further comprise one or more active agents selected from beta adrenergic agonists for simultaneous, sequential or separate use.

The present invention also provides the use of the pharmaceutical composition for the treatment of chronic obstructive pulmonary disease and asthma or related respiratory disorders.

The present invention also provides a pharmaceutical composition as substantially described herein by reference to the examples.

The following examples are for the purpose of illustration of the invention only and are not intended in any way to limit the scope of the present invention.

### EXAMPLE 1

| Sr. No. | Ingredients | Quantity/Spray |
|---|---|---|
| 1 | Tiotropium Bromide | 9 mcg |
| 2 | HFA/HFC-227 | q.s. to make up to 100 mcl |

Process:

1) Tiotropium bromide was homogenized with part quantity of HFA-227.

2) The suspension obtained in step (1) was transferred to a mixing vessel where the sufficient quantity of HFA-227 was added to make up the required volume of the can.

3) The resulting suspension was then mixed, recirculated and filled into pre-crimped Aluminium cans.

### EXAMPLE 2

| Sr. No. | Ingredients | Quantity/Spray |
|---|---|---|
| 1 | Tiotropium Bromide | 9 mcg |
| 2 | Lactose | 9 mcg |
| 3 | HFA/HFC-227 | q.s. to make up to 100 mcl |

Process:

1) Tiotropium bromide was homogenized with lactose and part quantity of HFA-227.

2) The suspension obtained in step (1) was transferred to the mixing vessel where sufficient quantity of HFA-227 was added to make up the required volume of the can.

3) The resulting suspension was then mixed, recirculated and filled into pre-crimped Aluminium cans.

### EXAMPLE 3

| Sr. No. | Ingredients | Quantity/Spray |
|---|---|---|
| 1 | Tiotropium Bromide | 9 mcg |
| 2 | PEG 1000 | 0.3% of total weight |
| | | of composition |
| 3 | PVP K 25 | 0.001% of total weight |
| | | of composition |
| 4 | Lactose | 9 mcg |
| 5 | HFA/HFC 227 | q.s. to make up to 100 mcl |

Process:

1) PEG and PVP were dissolved in HFA-227.

2) Tiotropium bromide was homogenized with lactose and part quantity of HFA-227.

3) The suspension obtained in step (2) was transferred to the mixing vessel where the sufficient quantity of HFA-227 was added to make up the required volume of the can.

4) The resulting suspension was then mixed, recirculated and filled into pre-crimped Aluminium cans.

The use of “q.s” in the tables above refers to the amount of the propellant which is required to make up the desired volume of the inhaler, excluding the volume of the other components stated.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including”, “comprising” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise.

1. A pharmaceutical composition comprising tiotropium, a hydrofluoroalkane (HFA) propellant, and optionally one or more pharmaceutically acceptable excipients.

2. A pharmaceutical composition according to claim 1, comprising tiotropium in the form of a pharmaceutically acceptable derivative thereof.

3. A pharmaceutical composition according to claim 2, wherein the pharmaceutically acceptable derivative is a salt, solvate, complex, hydrate, isomer, ester, tautomer, anhydrate, enantiomer, polymorph or prodrug.

4. A pharmaceutical composition according to claim 1, wherein the HFA propellant is HFA-227.

5. A pharmaceutical composition according to claim 1, wherein the tiotropium is in the form of tiotropium bromide.

6. A pharmaceutical composition according to claim 1, wherein the one or more pharmaceutically acceptable excipients is selected from HFA propellants, non-halogenated hydrocarbon propellants, co-solvents, low volatility components, stabilizers, dispersing agents, pH adjusting agents, antioxidants, preservatives, chelating agents, surface active agents, bulking agents, or mixtures thereof.

7. A pharmaceutical composition according to claim 1, further comprising one or more active agents selected from albuterol, salbutamol, levoalbuterol, levosalbutamol butaline, pirbuterol, procaterol, metaproterenol, fenoterol, isoproterenol (β₁ and β₂), metaproterenol, terbutaline, isoetarine, bitolterol mesylate, ritodrine, salmeterol, formoterol, arformoterol, carmoterol, bambuterol, clenbuterol, indacaterol, milveterol, vilanterol, olodaterol, fluticasone propionate, fluticasone furoate, fluticasone valerate, mometasone, ciclesonide, beclomethasone, budesonide, R-budesonide or their pharmaceutically acceptable derivatives thereof.

8. A pharmaceutical composition according to claim 7, wherein the pharmaceutically acceptable derivative is a salt, solvate, complex, hydrate, isomer, ester, tautomer, anhydrate, enantiomer, polymorph or prodrug.

9. A pharmaceutical composition according to claim 1, wherein the tiotropium is present in an amount from about 2.5 micrograms to about 18 micrograms.

10. A pharmaceutical composition according to claim 1, formulated for administration using a metered dose inhaler or a breath actuated metered dose inhaler.

11. A pharmaceutical composition according to claim 10, wherein the inhaler comprises a metering valve and low orifice actuator ranging from 0.2 mm to 0.6 mm diameter.

12. A process for preparing a pharmaceutical composition according to claim 1, which process comprises admixing tiotropium with a HFA propellant.

13. A process according to claim 12, wherein the admixing step comprises admixing one or more pharmaceutically acceptable excipients with tiotropium, and/or with a HFA propellant.

14. (canceled)

15. (canceled)

16. A method for the prophylaxis or treatment of asthma, chronic obstructive pulmonary disease and related respiratory disorders in a patient in need thereof, which method comprises administering a pharmaceutical composition according to claim 1.

17. A method according to claim 16, wherein the pharmaceutical composition is administered using a metered dose inhaler.

18. (canceled)
